(19) United States
(12) Patent Application Publication
(10) Pub. No.: US 2023/0094879 A1
(43) Pub. Date: Mar. 30, 2023

(54) GAS ANALYSIS SYSTEM BASED ON INTRINSICALLY SAFE GAS CHROMATOGRAPHY AND ITS METHOD OF USE

(71) Applicant: CCTEG Shenyang Research Institute, Fushun (CN)

(72) Inventors: Yuntao Liang, Fushun (CN); Junjie Zhang, Fushun (CN); Yong Sun, Fushun (CN); Jie Ren, Fushun (CN); Xuewei Ge, Fushun (CN); Wenjie Fang, Fushun (CN); Honghao He, Fushun (CN); Jilin Cui, Fushun (CN); Depeng Zhang, Fushun (CN); Cheng'ao Fu, Fushun (CN)

(21) Appl. No.: 17/484,211

(22) Filed: Sep. 24, 2021

(51) Int. Cl. G01N 30/32; G01N 33/00

(52) U.S. Cl. CPC G01N 30/32; G01N 33/0027; G01N 2030/324; G01N 2030/328; G01N 2030/025

(57) ABSTRACT

The disclosure includes a gas analysis system comprising a control system. In some embodiments, the control system comprises a computer monitoring host, an underground coal mine gas circuit control box including an intrinsically safe PLC, a sampling pump electrically coupled to the remote power control, an explosion proof safety power box electrically coupled to the intrinsically safe optical fiber switch, and an intrinsically safe gas chromatograph electrically coupled to the intrinsically safe optical fiber switch and the flameproof and intrinsically safe power box. In some embodiments, the gas analysis system comprises a gas pipeline system having an instrument sequence tube coupled to the carrier gas output pressure sensor, the carrier gas proportional solenoid valve, the carrier gas path pressure sensor, a manual carrier gas pressure reducing valve, and a carrier gas storage.

### BACKGROUND

### Field

The invention relates to a gas analysis system. In particular, the invention relates to a gas analysis system based on an intrinsically safe gas chromatograph that can be used in an explosive environment in a coal mine.

### Description of Related Art

Currently, equipment for gas analysis systems in coal mines is primarily used on the ground. Consequently, the gas at the detection location is extracted from the ground and transported through a long-distance transmission pipeline to the main analysis equipment, which may be far from the actual detection location. Some shortcomings in this approach are that it can result in added cost, complexity, possible contamination, and extra work for the labor crew.

### SUMMARY

The disclosure includes a gas analysis system comprising a control system. The control system may comprise a computer monitoring host on the ground electrically coupled to an optical terminal. According to some embodiments, the control system comprises an underground coal mine gas circuit control box including an intrinsically safe PLC (Programmable Logic Controller) electrically coupled to at least one of: an intrinsically safe optical fiber switch, a flameproof and intrinsically safe power supply box, a carrier gas output pressure sensor, a carrier gas proportional solenoid valve, a carrier gas path pressure sensor, a standard gas output pressure sensor, a standard gas proportional solenoid valve, a standard gas pressure sensor, a flow meter, a plurality of intrinsically safe solenoid valves, a plurality of pressure sensors, an intrinsically safe optical fiber switch optical cable coupled to an optical transceiver, a remote power control, and a remote power control switch. In some embodiments, the control system comprises a sampling pump electrically coupled to the remote power control. The control system may comprise an explosion proof safety power box electrically coupled to the intrinsically safe optical fiber switch. According to some embodiments, the control system comprises an intrinsically safe gas chromatograph electrically coupled to the intrinsically safe optical fiber switch and the flameproof and intrinsically safe power box.

In some embodiments, the gas analysis system comprises a gas pipeline system. The gas pipeline system may comprise an instrument sequence tube coupled to the carrier gas output pressure sensor, the carrier gas proportional solenoid valve, the carrier gas path pressure sensor, a manual carrier gas pressure reducing valve, and a carrier gas storage. According to some embodiments, the gas pipeline system may comprise having the intrinsically safe gas chromatograph coupled to at least one of the standard gas pressure sensor, the standard gas proportional solenoid valve, the standard gas output pressure sensor, a manual standard gas pressure reducing valve, a standard gas storage, the intrinsically safe gas chromatograph, a flow meter, at least one of the plurality of intrinsically safe solenoid valves, a filter, a manual flow regulating valve, a gas sampling port, the sampling pump, and a pressure sensor selecting from the group consisting of the carrier gas output pressure sensor, the carrier gas path pressure sensor, the standard gas output pressure sensor, the standard gas pressure sensor, and the plurality of pressure sensors.

In some embodiments, the gas analysis system comprises the underground coal mine gas circuit control box including the intrinsically safe PLC being electrically coupled to: the intrinsically safe optical fiber switch, the flameproof and intrinsically safe power supply box, the carrier gas output pressure sensor, the carrier gas proportional solenoid valve, the carrier gas path pressure sensor, the standard gas output pressure sensor, the standard gas proportional solenoid valve, the standard gas pressure sensor, the flow meter, the plurality of intrinsically safe solenoid valves, the plurality of pressure sensors, the intrinsically safe optical fiber switch optical cable coupled to the optical transceiver, the remote power control, and the remote power control switch.

In some embodiments, the gas analysis system comprises the intrinsically safe gas chromatograph coupled to the standard gas pressure sensor, the standard gas proportional solenoid valve, the standard gas output pressure sensor, the manual standard gas pressure reducing valve, the standard gas storage, the intrinsically safe gas chromatograph, the flow meter, at least one of the plurality of intrinsically safe solenoid valves, the filter, the manual flow regulating valve, the gas sampling port, the sampling pump, and the pressure sensor selecting from the group consisting of the carrier gas output pressure sensor, the carrier gas path pressure sensor, the standard gas output pressure sensor, the standard gas pressure sensor, and the plurality of pressure sensors.

According to some embodiments, the gas analysis system comprises five intrinsically safe solenoid valves, five pressure sensors, five filters, five manual flow regulating valves, and five gas sampling ports.

In some embodiments, the flameproof and intrinsically safe power supply box of the gas analysis system further comprises a power supply electrically coupled to a rechargeable battery. The rechargeable battery may be electrically coupled to the intrinsically safe gas chromatograph and the intrinsically safe optical fiber switch.

The disclosure also includes a method for using a gas analysis system. In some embodiments, the method comprises inputting a carrier gas output pressure sensor pressure comparison value T2 into a computer monitoring host. In some embodiments, the method comprises inputting a carrier gas path pressure sensor pressure comparison value T6 into the computer monitoring host. According to some embodiments, the method comprises inputting a standard gas output pressure sensor pressure measurement comparison value T3 into the computer monitoring host. In some embodiments, the method comprises inputting a standard gas path pressure sensor pressure measurement comparison value T7 into the computer monitoring host. In some embodiments, the method comprises inputting a pressure sensor pressure measurement comparison value T′ into the computer monitoring host.

According to some embodiments, the method comprises manually opening a manual carrier gas pressure reducing valve. In some embodiments, the method comprises manually opening a standard gas pressure reducing valve. The method may comprise calculating, via the carrier gas output pressure sensor, a carrier gas output pressure T0. According to some embodiments, the method comprises comparing a carrier output pressure T0 to the carrier gas output pressure sensor pressure comparison value T2. In some embodiments, the method comprises, in response to the carrier output pressure T0 being less than or equal to the carrier gas output pressure sensor pressure comparison value T2, determining that the pressure in a carrier gas storage is insufficient, alarming the carrier gas storage, and replacing the carrier gas storage.

In some embodiments, the method comprises calculating, via the carrier gas pressure sensor, a carrier gas pressure T4. According to some embodiments, the method comprises comparing the carrier gas pressure T4 to the carrier gas path pressure sensor pressure comparison value T6. In some embodiments, the method comprises, in response to the carrier gas pressure T4 being not equal to the carrier gas path pressure sensor pressure comparison value T6, automatically adjusting, via an intrinsically safe PLC, a carrier gas ratio such that the carrier gas pressure T4 is equal to the carrier gas path pressure sensor pressure comparison value T6.

In some embodiments, the method comprises calculating, via the standard gas output pressure sensor, a standard gas output pressure T1. According to some embodiments, the method comprises comparing the standard gas output pressure T1 to the standard gas output pressure sensor pressure measurement comparison value T3. In some embodiments, the method comprises, in response to the standard gas output pressure T1 being less than or equal to the standard gas output pressure sensor pressure measurement comparison value T3, determining that the pressure in the standard gas storage is insufficient, alarming the standard gas storage, and replacing the standard gas storage.

In some embodiments, the method comprises calculating, via the standard gas circuit pressure sensor, a standard gas circuit pressure T5. According to some embodiments, the method comprises comparing the standard gas circuit pressure T5 to the standard gas path pressure sensor pressure measurement comparison value T7. In some embodiments, the method comprises, in response to the pressure measurement value of the standard gas circuit pressure T5 being not equal to the standard gas path pressure sensor pressure measurement comparison value T7, automatically adjusting, via the intrinsically safe PLC, a standard gas proportional solenoid valve opening such that the pressure measurement value of the standard gas circuit pressure T5 is equal to the standard gas path pressure sensor pressure measurement comparison value T7. In some embodiments, the method comprises turning on a remote power control switch from the computer monitoring host. According to some embodiments, the method may comprise turning on a sampling pump. In some embodiments, the method comprises manually adjusting a manual flow control valve to make a flow value L equal to a set point within a range of values L0.

According to some embodiments, the method comprises calculating, via the pressure sensor, a pressure measurement value T. According to some embodiments, the method comprises comparing the pressure measurement value T to the pressure sensor pressure measurement comparison value T′. In some embodiments, the method comprises, in response to the pressure measurement value of the pressure measurement value T being less than or equal to the pressure sensor pressure measurement comparison value T′, determining that the pressure in a gas transmission pressure is insufficient, alarming the sampling pump, and replacing the sampling pump.

According to some embodiments, the method comprises selecting a calibration option on the computer monitoring host. In some embodiments, the method comprises setting an intrinsically safe gas chromatograph to calibration status.

According to some embodiments, the method comprises selecting a gas sampling port corresponding to any pipeline to be analyzed from the computer monitoring host. In some embodiments, the method comprises opening an intrinsically safe solenoid valve. The method may comprise analyzing, via the intrinsically safe gas chromatograph, the sample gas. According to some embodiments, the method comprises automatically opening another intrinsically safe solenoid valve corresponding to the gas sampling port. In some embodiments, the method comprises analyzing another sample gas until all gas in the gas sampling port is analyzed. The method may comprise reading a flowmeter reading from the computer monitoring host at any time. According to some embodiments, the method comprises tracking a process.

In some embodiments, the method for using a gas analysis system comprises selecting the carrier gas output pressure sensor pressure comparison value T2 from a range of 1.5 Mpa to 2.5 Mpa. The method for using a gas analysis system may comprise selecting the carrier gas path pressure sensor pressure comparison value T6 from a range of 0.3 Mpa to 0.5 Mpa. According to some embodiments, the method for using a gas analysis system comprises selecting the standard gas output pressure sensor pressure measurement comparison value T3 from a range of 1.5 Mpa to 2.5 Mpa. In some embodiments, the method for using a gas analysis system comprises selecting the standard gas path pressure sensor pressure measurement comparison value T7 from a range of 0.02 Mpa to 0.2 Mpa. In some embodiments, the method for using a gas analysis system comprises selecting the pressure sensor pressure measurement comparison value T′ from a range of 0.002 Mpa to 0.01 Mpa. According to some embodiments, the method for using a gas analysis system comprises selecting the range of values L0 from a range of 0.5 L/min to 8 L/min.

In some embodiments, the method for using a gas analysis system comprises selecting the carrier gas output pressure sensor pressure comparison value T2 to be 2 Mpa. In some embodiments, the method for using a gas analysis system comprises selecting the carrier gas path pressure sensor pressure comparison value T6 to be 0.4 Mpa. According to some embodiments, the method for using a gas analysis system comprises selecting the standard gas output pressure sensor pressure measurement comparison value T3 to be 2 Mpa. In some embodiments, the method for using a gas analysis system comprises selecting the standard gas path pressure sensor pressure measurement comparison value T7 to be 0.1 Mpa. According to some embodiments, the method for using a gas analysis system comprises selecting the pressure sensor pressure measurement comparison value T′ to be 0.005 Mpa.

### BRIEF DESCRIPTION OF THE DRAWINGS

These and other features, aspects, and advantages are described below with reference to the drawings, which are intended to illustrate, but not to limit, the embodiments disclosed. In the drawings, like reference characters denote corresponding features consistently throughout similar embodiments.

FIG. 1 is a schematic structural diagram, according to some embodiments.

FIG. 2 is a schematic diagram of the structure of a gas circuit control box, according to some embodiments.

FIG. 3 illustrates a flow chart depicting a method of using a gas analysis system.

FIG. 4 illustrates a flow chart depicting a method of comparing and responding to a carrier gas output pressure.

FIG. 5 illustrates a flow chart depicting a method of comparing and responding to a carrier gas pressure.

FIG. 6 illustrates a flow chart depicting a method of comparing and responding to a standard gas output pressure.

FIG. 7 illustrates a flow chart depicting a method of comparing and responding to a standard gas path pressure.

FIG. 8 illustrates a flow chart depicting a method of comparing and responding to a pressure measurement value.

FIG. 9 illustrates a flow chart depicting a method of calibrating a computer monitoring host.

FIG. 10 illustrates a flow chart depicting a method of analyzing gas samples.

FIG. 11 illustrates a flow chart depicting a method of selecting range values for various pressures and flow rates.

FIG. 12 illustrates a flow chart depicting a method of selecting precise values for various pressures.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### COMPONENT INDEX

1—Carrier gas storage
2—Standard gas storage
3—Manual carrier gas pressure reducing valve
4—Manual standard gas pressure reducing valve
5—Carrier gas output pressure sensor
6—Carrier gas proportional solenoid valve
7—Standard gas output pressure sensor
8—Standard gas proportional solenoid valve
9—Intrinsically safe gas chromatograph
10—Remote power control switch
11—Sampling pump
12—Flameproof and intrinsically safe power box
13—Computer monitoring host
14—Intrinsically safe optical fiber switch
15—Gas control box
16—Optical transceiver
17—Carrier gas pressure sensor
18—Standard gas pressure sensor
151—Intrinsically safe PLC
152—Flowmeter
152—Intrinsically safe solenoid valve
154—Pressure sensor
155—Filter
156—Manual flow control valve
157—Gas sampling port

One problem with the prior art includes the gas delivery pipeline being too long, resulting in long sampling times and facilitating contamination of the gas components during its travel through the pipeline. Another problem with the prior art is that the large amount of maintenance required creates a greater workload for the crew. A third issue in the prior art is that the equipment is scattered, and controlling the equipment remotely is not feasible. A final issue with the prior art is that conventional analysis equipment is complicated to use and requires a professional operator.

In addition, according to Article 14 of the document Anbiaozi [2016] No. 35 of the Anbiao National Mining Product Safety Marking Center document, “When hazardous gas monitoring equipment (such as underground beam tube detection equipment) is used, the overall structure cannot meet the explosion-proof requirements, and there is the possibility of dangerous gas leakage in the equipment cavity, so explosion-proof design should not be adopted.” According to this regulation, intrinsically safe equipment must be used in hazardous gas environment monitoring equipment, and explosion-proof equipment is not feasible and thus should not be used.

Many embodiments disclosed relate to a gas analysis system based on intrinsically safe gas chromatography, containing a control system and a gas pipeline system. The control system includes a computer on the ground monitoring host power and connecting to an optical transceiver, which is characterized by the control system including an intrinsically safe PLC inside the control box of the gas pipeline system underground in a coal mine, and the intrinsically safe PLC electrically coupling to an intrinsic optical switch, among other controlled parts. The gas pipeline system may contain pressure sensors on the gas pipelines coupling the intrinsic gas chromatograph in sequence with other parts. An application method for use of the gas analysis system based on an intrinsic safe chromatograph is also claimed. The disclosed embodiments allow for the use of this application in dangerous gas environments, which have the potential to become explosive under certain conditions. The method is also portable and easy to use with broad usages in dangerous gas environments through the use of either remote or local controlling. The embodiments may realize remote control and data transfer. Disclosed embodiments may also solve the issues of real-time lag caused by remote gas sampling, heavy maintenance workload, and large data errors.

In order to solve the above problems and meet the requirements for the use of hazardous gas detection equipment of the National Mining Product Safety Marking Center of Safety Standards, the disclosed embodiments provide an intrinsically safe gas chromatograph that can be used in an explosive atmosphere.

The technical solution adopted by the disclosed embodiments is a gas analysis system based on an intrinsically safe gas chromatograph, composed of a control system and a gas pipeline system. The control system includes a computer monitoring host on the ground that is electrically coupled to an optical transceiver. The control system also includes an intrinsically safe PLC installed in the underground gas circuit control box of the coal mine. The intrinsically safe PLC is electrically coupled to the intrinsically safe optical fiber switch, the flameproof and intrinsically safe power box, the carrier gas output pressure sensor, the carrier gas proportional solenoid valve, and the carrier gas. The control system may also include a gas pressure sensor, standard output pressure sensor, standard gas proportional solenoid valve, standard gas pressure sensor, flow meter, several intrinsically safe solenoid valves, several pressure sensors, intrinsically safe optical fiber switch, and an optical cable coupled to an optical transceiver. The optical fiber switch is also electrically coupled to the flameproof and intrinsically safe power supply box and the remote power control switch. The remote power control switch is electrically coupled to the sampling pump. The intrinsically safe gas chromatograph is electrically coupled to the intrinsically safe optical fiber switch and the flameproof and intrinsically safe power supply box. The gas pipeline system includes an intrinsically safe gas chromatograph sequence tube coupled to a carrier gas pressure sensor, a carrier gas proportional solenoid valve, a carrier gas output pressure sensor, a manual carrier gas pressure reducing valve, a carrier gas storage, and an intrinsically safe gas chromatograph. The sequence tube is also coupled to the standard gas pressure sensor, the standard gas proportional solenoid valve, the standard gas output pressure sensor, the manual standard gas pressure reducing valve, and the standard gas storage. The intrinsically safe gas chromatograph is also coupled to the flowmeter and intrinsically safe solenoid valve, pressure sensor, filter, manual flow control valve, gas sampling port, and sampling pump.

The beneficial effects of the disclosed embodiments include utilizing the system in a dangerous gas environment. Additionally, it may increase convenience, increase the application range in the dangerous area, and result in the ability to carry out the monitoring and analysis remotely and/or on-site. The application can allow for customized services, permit remote system operation and remote transmission of data, and solve the problems of real-time lag caused by remote sampling, large maintenance workload, and large data analysis errors.

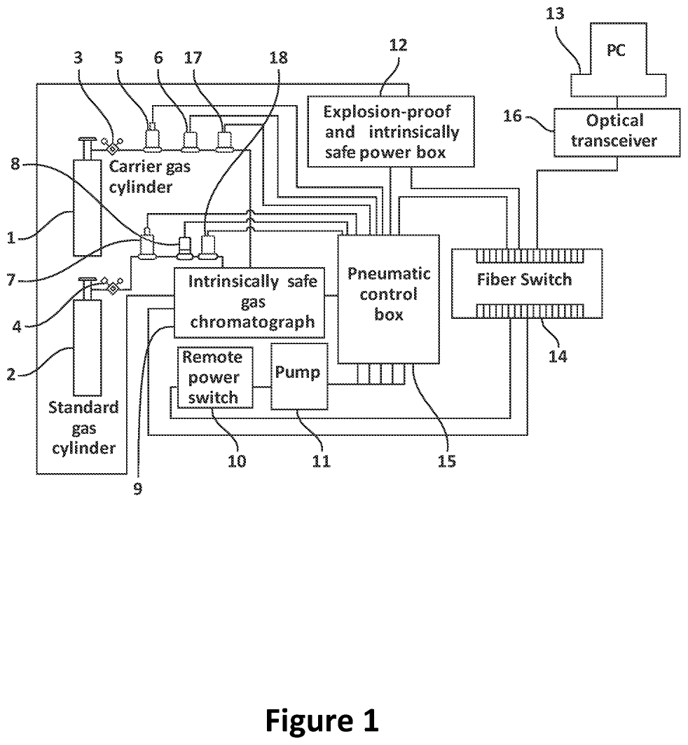

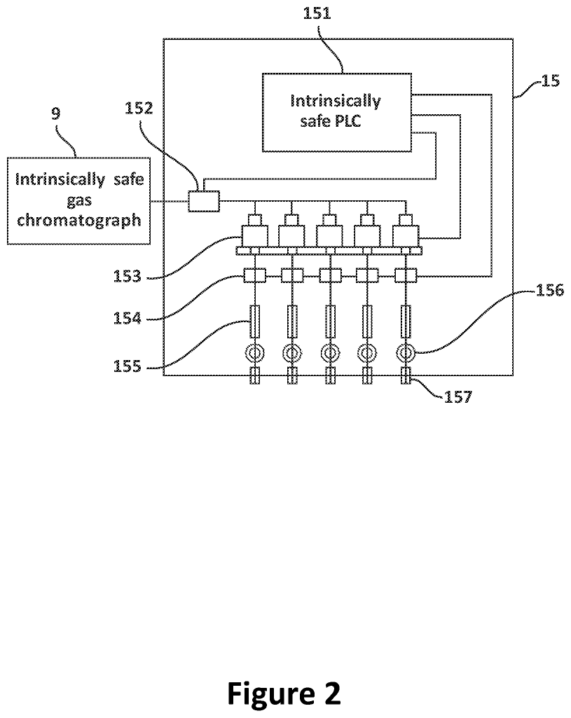

FIGS. 1 and 2 illustrate an embodiment of a gas analysis system based on an intrinsically safe chromatograph with a control system and a gas pipeline system. The control system may comprise a computer monitoring host 13 on the ground, which is electrically coupled to an optical transceiver 16, and is characterized in that the control system also includes underground coal mine gas. According to some embodiments, intrinsically safe PLC 151 is installed in the control box 15, which is electrically coupled to the intrinsically safe fiber switch 14, the flameproof and intrinsically safe power box 12, the carrier gas output pressure sensor 5, the carrier gas proportional solenoid valve 6, and the carrier gas pressure sensor 17.

According to some embodiments, the gas analysis system has a standard gas output pressure sensor 7, a standard gas proportional solenoid valve 8, a standard gas path pressure sensor 18, a flow meter 152, several intrinsically safe solenoid valves 153, several pressure sensors 154, and an intrinsically safe optical fiber switch 14. The optical cable may be coupled to the optical transceiver 16. In some embodiments, the intrinsically safe optical fiber switch 14 is electrically coupled to the flameproof and intrinsically safe power supply box 12 and the remote power control switch 10. The remote power control switch 10 may be electrically coupled to the sampling pump 11. The intrinsically safe gas chromatograph 9 may be electrically coupled to the optical fiber switch 14, an explosion-proof and intrinsically safe power supply box 12, and the remote power control switch 10. In some embodiments, the remote power control switch 10 is electrically coupled to the sampling pump 11, and the intrinsically safe gas chromatograph 9 is electrically coupled to the optical fiber switch 14 and an explosion-proof and intrinsically safe power supply box 12. The gas pipeline system may include an intrinsically safe gas chromatograph 9 and have sequential pipes coupled to the carrier gas pressure sensor 17, the carrier gas proportional solenoid valve 6, the carrier gas output pressure sensor 5, the manual carrier gas pressure reducing valve 3, and the carrier gas storage 1. According to some embodiments, the intrinsically safe gas chromatograph 9 is also coupled to the standard gas circuit pressure sensor 18, the standard gas proportional solenoid valve 8, the standard gas output pressure sensor 7, the manual standard gas pressure reducing valve 4, the standard gas storage 2, and the intrinsically safe gas chromatograph 9. The intrinsically safe gas chromatograph 9 may be coupled to the flowmeter 152, the intrinsically safe solenoid valve 153, the pressure sensor 154, the filter 155, the manual flow control valve 156, the gas sampling port 157, and the sampling pump 11. The sampling pump 11 may be selected as KGS-120.

FIGS. 1 and 2 also illustrate an embodiment of a gas analysis system where the flameproof and intrinsically safe power supply box 12 includes a power supply that is electrically coupled to a rechargeable battery, and the rechargeable battery is electrically coupled to the intrinsically safe gas chromatograph 9 and the intrinsically safe optical fiber switch 14. The method of using a gas analysis system based on an intrinsically safe gas chromatograph may be mainly implemented by the intrinsically safe PLC 151 after programming.

FIGS. 1 and 2 further illustrate a method of using a gas analysis system based on an intrinsically safe gas chromatograph which features preparation, calibration, and analysis. The preparation phase may include inputting the carrier gas output pressure sensor 5 into the computer monitoring host 13 to measure the pressure comparison value T2, inputting the carrier gas path pressure sensor 17 into the computer monitoring host 13 to measure the pressure comparison value T6, inputting the standard gas output pressure sensor 7 into the computer monitoring host 13 to measure the pressure comparison value T3, inputting the standard gas path pressure sensor 18 into the computer monitoring host 13 to measure the pressure comparison value T7, inputting the pressure sensor 154 into the computer monitoring host 13 to measure the pressure comparison value T′, manually opening the manual carrier gas pressure reducing valve 3, and manually opening a standard gas pressure reducing valve 4. In some embodiments, the preparation includes calculating, via the carrier gas output pressure sensor 5, a carrier gas output pressure T0 and comparing it to T2, when T0 is less than or equal to T2, it is determined that the pressure in the carrier gas storage 1 in insufficient, an alarm is issued, and the carrier gas storage 1 is replaced. According to some embodiments, the preparation includes calculating, via the carrier gas circuit pressure sensor 17, the carrier gas pressure T4 and comparing it to T6, when T4 is not equal to T6, the intrinsically safe PLC 151 automatically adjusts the opening degree of the carrier gas proportional solenoid valve 6 to make T4 equal to T6. The preparation may include calculating, via the standard gas output pressure sensor 7, the standard gas output pressure T1 and comparing it to T3, when T1 is less than or equal to T3, it is determined that the pressure in the standard gas storage 2 is insufficient, an alarm is issued, and the standard gas storage 2 is replaced. In some embodiments, the preparation includes calculating, via the standard gas path pressure sensor 18, the standard gas circuit pressure T5 and comparing it to T7, when T5 is not equal to T7, the intrinsically safe PLC 151 automatically adjusts the opening degree of the standard gas proportional solenoid valve 8 to make T5 equal to T7, the remote power control switch 10 is turned on from the computer monitoring host 13, the sampling pump 11 is turned on, and the manual flow regulating valve 156 is manually adjusted to make the flow value L within the range of the set value L0. According to some embodiments, the preparation includes calculating, via pressure sensor 154, the pressure T and comparing it to T′, when T is less than or equal to T′, determining that the gas delivery pressure is insufficient, an alarm is issued, and the sampling pump is replaced.

In some embodiments, the selection range for T2 is 1.5 Mpa to 2.5 Mpa, the selection range for T6 is 0.3 Mpa to 0.5 Mpa, the selection range for T3 is 1.5 Mpa to 2.5 Mpa, the selection range for T7 is 0.02 Mpa to 0.2 Mpa, the selection range for T′ is 0.002 Mpa to 0.01 Mpa, and the selection range for L0 is 0.5 L/min to 8 L/min. According to some embodiments, the manual flow control valve 156 selects the LZB-6WB model that displays flow.

According to some embodiments, T2 is 2 Mpa, T6 is 0.4 Mpa, T3 is 2 Mpa, T7 is 0.1 Mpa, and T′ is 0.005 Mpa.

The calibration phase may include selecting the calibration option on the computer monitoring host 13 and setting the intrinsically safe gas chromatograph 9 to the calibration state.

The analysis phase may include selecting any pipeline corresponding to the computer monitoring host 13 that needs to be analyzed. According to some embodiments, the analysis includes opening the intrinsically safe solenoid valve 153 of the gas sampling port 157. After the intrinsically safe gas chromatograph 9 analyzes the sample gas, it may automatically open another intrinsically safe solenoid valve 153 corresponding to the gas sampling port 157. In some embodiments, the gas is analyzed until all of the gas at the gas sampling port 157 is analyzed. According to some embodiments, during the analysis, the reading of the flowmeter 152 can be read from the computer monitoring host 13 at any time to track the process.

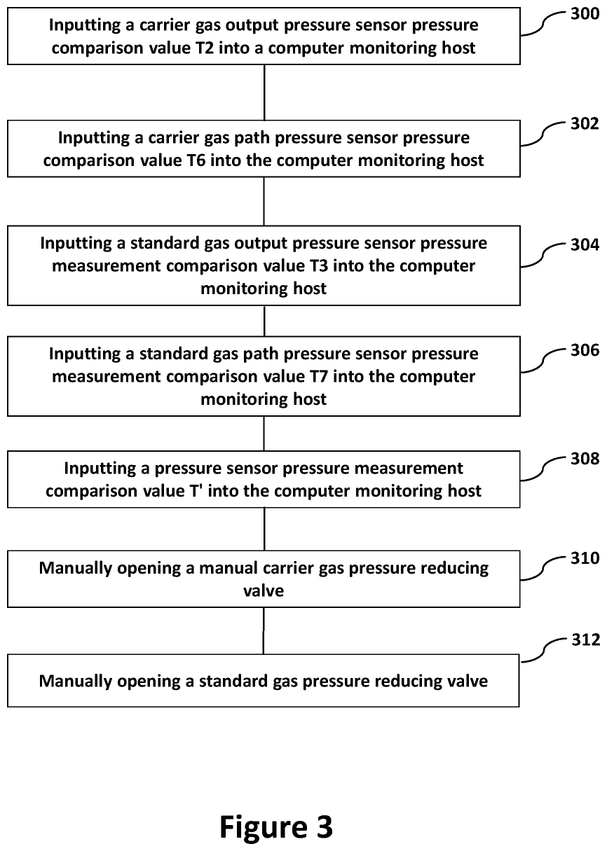

FIG. 3 illustrates a method of using a gas analysis system. The method may include inputting a carrier gas output pressure sensor pressure comparison value T2 into a computer monitoring host (at step 300). According to some embodiments, the method includes inputting a carrier gas path pressure sensor pressure comparison value T6 into the computer monitoring host (at step 302). In some embodiments, the method includes inputting a standard gas output pressure sensor pressure measurement comparison value T3 into the computer monitoring host (at step 304). The method may include inputting a standard gas circuit pressure sensor pressure measurement comparison value T7 into the computer monitoring host (at step 306). According to some embodiments, the method includes inputting a pressure sensor pressure measurement comparison value T′ into the computer monitoring host (at step 308). In some embodiments, the method includes manually opening a manual carrier gas pressure reducing valve (at step 310). The method may include manually opening a standard gas pressure reducing valve (at step 312).

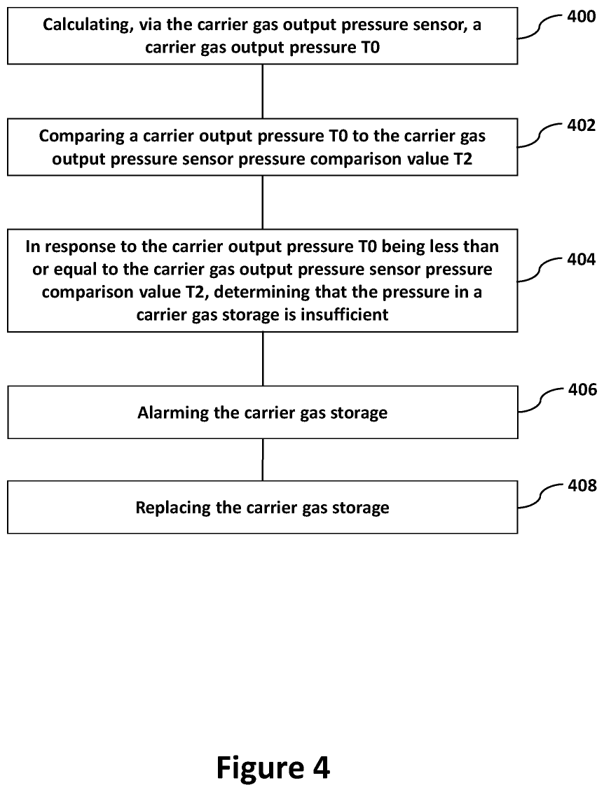

FIG. 4 illustrates a method of comparing and responding to a carrier gas output pressure. The method may include calculating, via the carrier gas output pressure sensor, a carrier gas output pressure T0 (at step 400). According to some embodiments, the method includes comparing a carrier output pressure T0 to the carrier gas output pressure sensor pressure comparison value T2 (at step 402). In some embodiments, the method includes, in response to the carrier output pressure T0 being less than or equal to the carrier gas output pressure sensor pressure comparison value T2, determining that the pressure in a carrier gas storage is insufficient (at step 404). The method may include alarming the carrier gas storage (at step 406). According to some embodiments, the method includes replacing the carrier gas storage (at step 408).

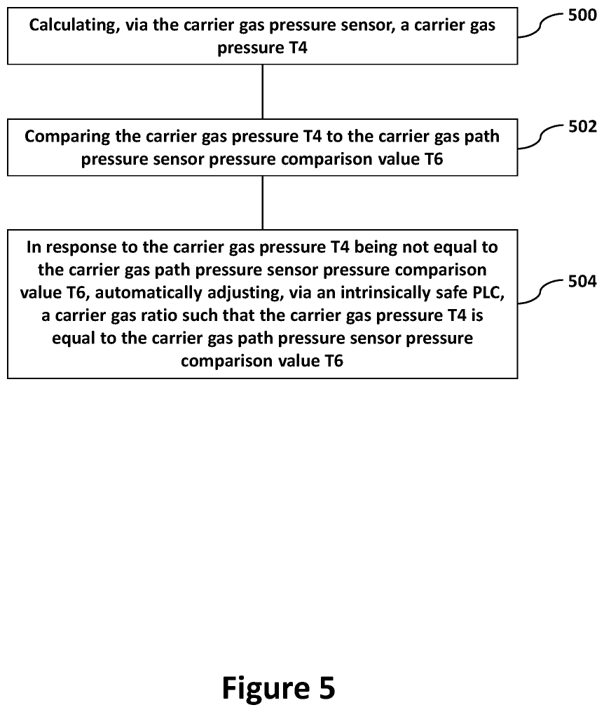

FIG. 5 illustrates a method of comparing and responding to a carrier gas pressure. The method may include calculating, via the carrier gas pressure sensor, a carrier gas pressure T4 (at step 500). According to some embodiments, the method includes comparing the carrier gas pressure T4 to the carrier gas path pressure sensor pressure comparison value T6 (at step 502). In some embodiments, in response to the carrier gas pressure T4 being not equal to the carrier gas path pressure sensor pressure comparison value T6, automatically adjusting, via an intrinsically safe PLC, a carrier gas ratio such that the carrier gas pressure T4 is equal to the carrier gas path pressure sensor pressure comparison value T6 (at step 504).

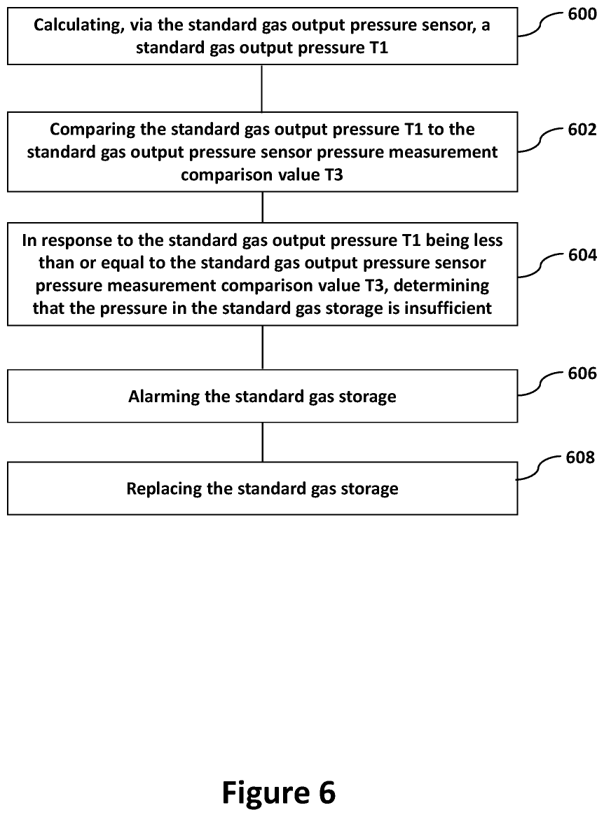

FIG. 6 illustrates a method of comparing and responding to a standard gas output pressure. The method may include calculating, via the standard gas output pressure sensor, a standard gas output pressure T1 (at step 600). According to some embodiments, the method includes comparing the standard gas output pressure T1 to the standard gas output pressure sensor pressure measurement comparison value T3 (at step 602). The method may include, in response to the standard gas output pressure T1 being less than or equal to the standard gas output pressure sensor pressure measurement comparison value T3, determining that the pressure in the standard gas storage is insufficient (at step 604). According to some embodiments, the method includes alarming the standard gas storage (at step 606). In some embodiments, the method includes replacing the standard gas storage (at step 608).

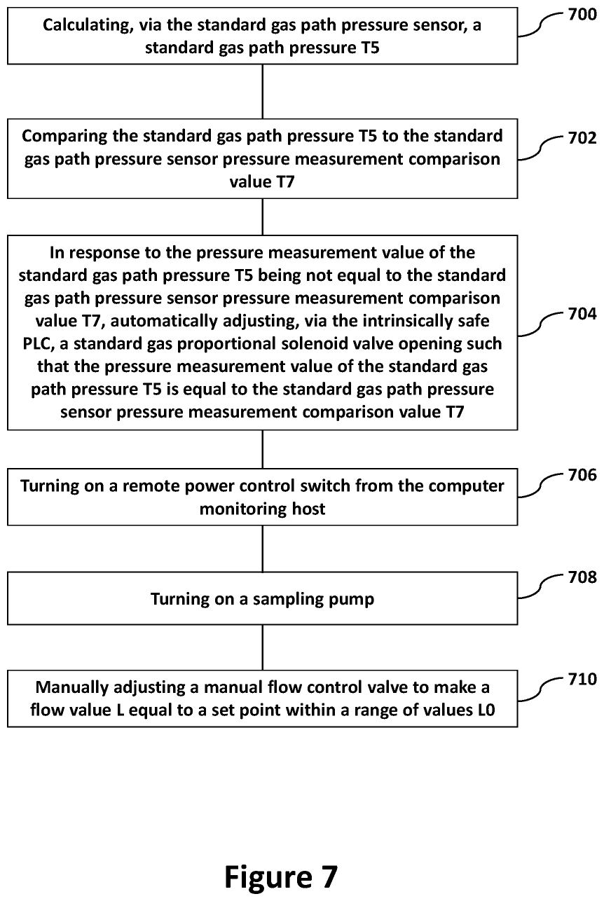

FIG. 7 illustrates a method of comparing and responding to a standard gas path pressure. The method may include calculating, via the standard gas circuit pressure sensor, a standard gas circuit pressure T5 (at step 700). According to some embodiments, the method includes comparing the standard gas circuit pressure T5 to the standard gas path pressure sensor pressure measurement comparison value T7 (at step 702). In some embodiments, the method includes, in response to the pressure measurement value of the standard gas circuit pressure T5 being not equal to the standard gas path pressure sensor pressure measurement comparison value T7, automatically adjusting, via the intrinsically safe PLC, a standard gas proportional solenoid valve opening such that the pressure measurement value of the standard gas circuit pressure T5 is equal to the standard gas path pressure sensor pressure measurement comparison value T7 (at step 704). The method may include turning on a remote power control switch from the computer monitoring host (at step 706). According to some embodiments, the method includes turning on a sampling pump (at step 708). In some embodiments, the method includes manually adjusting a manual flow control valve to make a flow value L equal to a set point within a range of values L0 (at step 710).

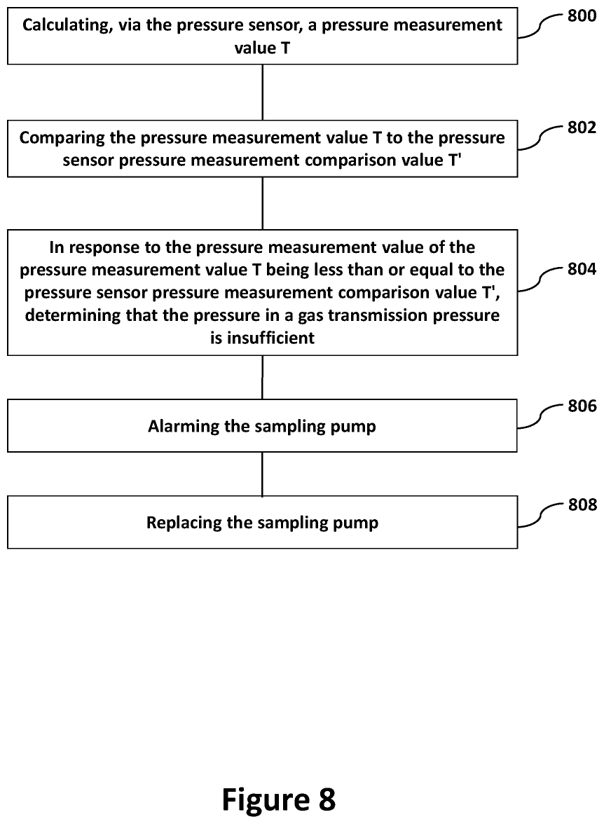

FIG. 8 illustrates a method of comparing and responding to a pressure measurement value. The method may include calculating, via the pressure sensor, a pressure measurement value T (at step 800). According to some embodiments, the method includes comparing the pressure measurement value T to the pressure sensor pressure measurement comparison value T′ (at step 802). In some embodiments, the method includes, in response to the pressure measurement value of the pressure measurement value T being less than or equal to the pressure sensor pressure measurement comparison value T′, determining that the pressure in a gas transmission pressure is insufficient (at step 804). The method may include alarming the sampling pump (at step 806). According to some embodiments, the method includes replacing the sampling pump (at step 808).

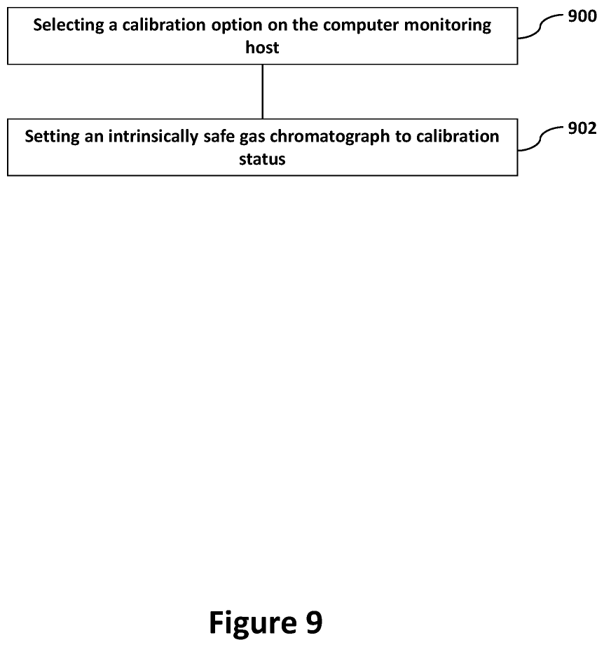

FIG. 9 illustrates a method of calibrating a computer monitoring host. The method may include selecting a calibration option on the computer monitoring host (at step 900). According to some embodiments, the method includes setting an intrinsically safe gas chromatograph to calibration status (at step 902).

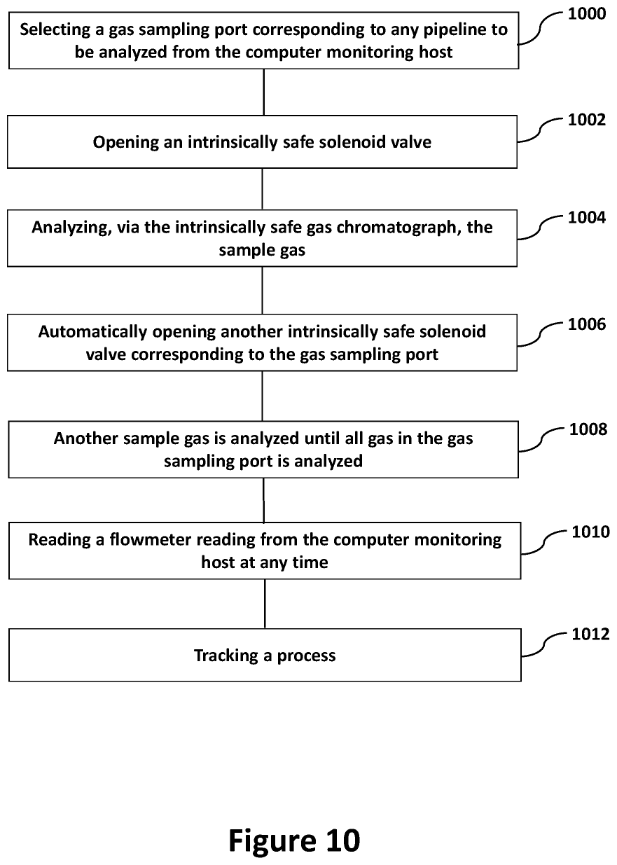

FIG. 10 illustrates a method of analyzing gas samples. The method may include selecting a gas sampling port corresponding to any pipeline to be analyzed from the computer monitoring host (at step 1000). According to some embodiments, the method includes opening an intrinsically safe solenoid valve (at step 1002). In some embodiments, the method includes analyzing, via the intrinsically safe gas chromatograph, the sample gas (at step 1004). The method may include automatically opening another intrinsically safe solenoid valve corresponding to the gas sampling port (at step 1006). According to some embodiments, the method includes analyzing another sample gas until all gas in the gas sampling port is analyzed (at step 1008). In some embodiments, the method includes reading a flowmeter reading from the computer monitoring host at any time (at step 1010). The method may include tracking a process (at step 1012).

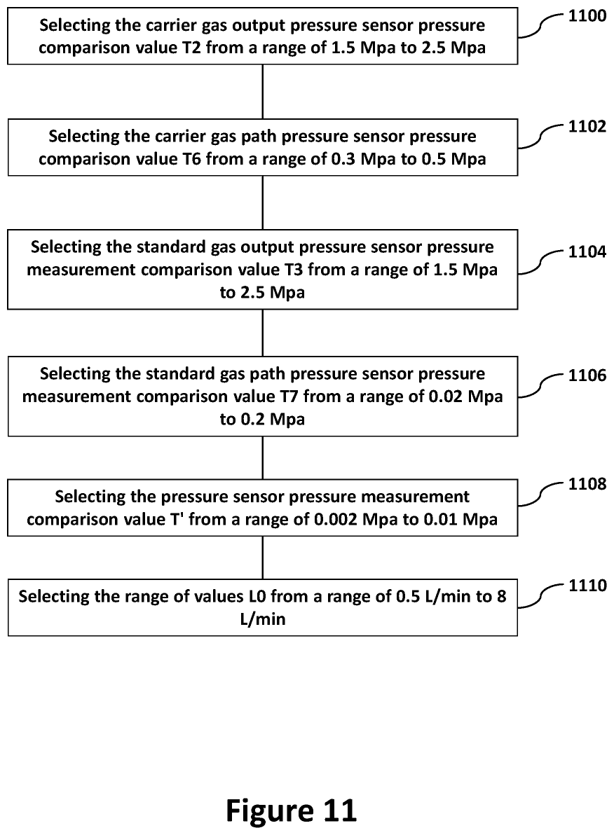

FIG. 11 illustrates a method of selecting range values for various pressures and flow rates. The method may include selecting the carrier gas output pressure sensor pressure comparison value T2 from a range of 1.5 Mpa to 2.5 Mpa (at step 1100). According to some embodiments, the method includes selecting the carrier gas path pressure sensor pressure comparison value T6 from a range of 0.3 Mpa to 0.5 Mpa (at step 1102). In some embodiments, the method includes selecting the standard gas output pressure sensor pressure measurement comparison value T3 from a range of 1.5 Mpa to 2.5 Mpa (at step 1104). The method may include selecting the standard gas path pressure sensor pressure measurement comparison value T7 from a range of 0.02 Mpa to 0.2 Mpa (at step 1106). According to some embodiments, the method includes selecting the pressure sensor pressure measurement comparison value T′ from a range of 0.002 Mpa to 0.01 Mpa (at step 1108). In some embodiments, the method includes selecting the range of values L0 from a range of 0.5 L/min to 8 L/min (at step 1110).

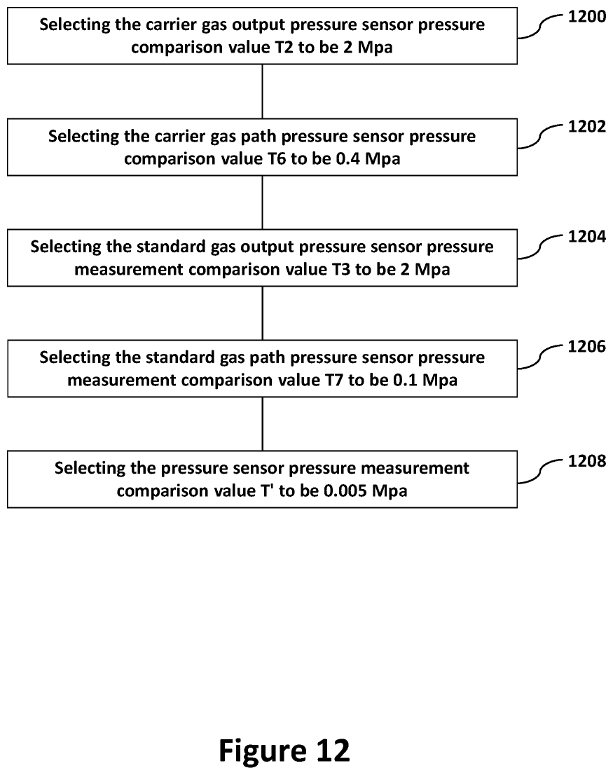

FIG. 12 illustrates a method of selecting precise values for various pressures. The method may include selecting the carrier gas output pressure sensor pressure comparison value T2 to be 2 Mpa (at step 1200). According to some embodiments, the method includes selecting the carrier gas path pressure sensor pressure comparison value T6 to be 0.4 Mpa (at step 1202). In some embodiments, the method includes selecting the standard gas output pressure sensor pressure measurement comparison value T3 to be 2 Mpa (at step 1204). The method may include selecting the standard gas path pressure sensor pressure measurement comparison value T7 to be 0.1 Mpa (at step 1206). According to some embodiments, the method includes selecting the pressure sensor pressure measurement comparison value T′ to be 0.005 Mpa (at step 1208).

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

To increase the clarity of various features, other features are not labeled in each figure.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the embodiments disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the embodiments disclosed herein.

What is claimed is:

1. A gas analysis system, comprising:
a control system including: a computer monitoring host on the ground electrically coupled to an optical terminal; an underground coal mine gas circuit control box including an intrinsically safe PLC electrically coupled to at least one of: an intrinsically safe optical fiber switch, a flameproof and intrinsically safe power supply box, a carrier gas output pressure sensor, a carrier gas proportional solenoid valve, a carrier gas path pressure sensor, a standard gas output pressure sensor, a standard gas proportional solenoid valve, a standard gas pressure sensor, a flow meter, a plurality of intrinsically safe solenoid valves, a plurality of pressure sensors, an intrinsically safe optical fiber switch optical cable coupled to an optical transceiver, a remote power control, and a remote power control switch; a sampling pump electrically coupled to the remote power control; an explosion proof safety power box electrically coupled to the intrinsically safe optical fiber switch; and an intrinsically safe gas chromatograph electrically coupled to the intrinsically safe optical fiber switch and the flameproof and intrinsically safe power box; and
a gas pipeline system including: an instrument sequence tube coupled to the carrier gas output pressure sensor, the carrier gas proportional solenoid valve, the carrier gas path pressure sensor, a manual carrier gas pressure reducing valve, and a carrier gas storage; and the intrinsically safe gas chromatograph coupled to at least one of the standard gas pressure sensor, the standard gas proportional solenoid valve, the standard gas output pressure sensor, a manual standard gas pressure reducing valve, a standard gas storage, the intrinsically safe gas chromatograph, a flow meter, at least one of the plurality of intrinsically safe solenoid valves, a filter, a manual flow regulating valve, a gas sampling port, the sampling pump, and a pressure sensor selecting from the group consisting of the carrier gas output pressure sensor, the carrier gas path pressure sensor, the standard gas output pressure sensor, the standard gas pressure sensor, and the plurality of pressure sensors.

2. The gas analysis system of claim 1, wherein the underground coal mine gas circuit control box including the intrinsically safe PLC is electrically coupled to the intrinsically safe optical fiber switch, the flameproof and intrinsically safe power supply box, the carrier gas output pressure sensor, the carrier gas proportional solenoid valve, the carrier gas path pressure sensor, the standard gas output pressure sensor, the standard gas proportional solenoid valve, the standard gas pressure sensor, the flow meter, the plurality of intrinsically safe solenoid valves, the plurality of pressure sensors, the intrinsically safe optical fiber switch optical cable coupled to the optical transceiver, the remote power control, and the remote power control switch.

3. The gas analysis system of claim 2, wherein the intrinsically safe gas chromatograph is coupled to the standard gas pressure sensor, the standard gas proportional solenoid valve, the standard gas output pressure sensor, the manual standard gas pressure reducing valve, the standard gas storage, the intrinsically safe gas chromatograph, the flow meter, at least one of the plurality of intrinsically safe solenoid valves, the filter, the manual flow regulating valve, the gas sampling port, the sampling pump, and the pressure sensor selecting from the group consisting of the carrier gas output pressure sensor, the carrier gas path pressure sensor, the standard gas output pressure sensor, the standard gas pressure sensor, and the plurality of pressure sensors.

4. The gas analysis system of claim 3, further comprising:
five intrinsically safe solenoid valves;
five pressure sensors;
five filters;
five manual flow regulating valves; and
five gas sampling ports.

5. The gas analysis system of claim 3, the flameproof and intrinsically safe power supply box further comprising a power supply electrically coupled to a rechargeable battery,
wherein the rechargeable battery is electrically coupled to the intrinsically safe gas chromatograph and the intrinsically safe optical fiber switch.

6. The gas analysis system of claim 3, further comprising:
five intrinsically safe solenoid valves;
five pressure sensors;
five filters;
five manual flow regulating valves; and
five gas sampling ports.

7. A method for using a gas analysis system, comprising:
inputting a carrier gas output pressure sensor pressure comparison value T2 into a computer monitoring host;
inputting a carrier gas path pressure sensor pressure comparison value T6 into the computer monitoring host;
inputting a standard gas output pressure sensor pressure measurement comparison value T3 into the computer monitoring host;
inputting a standard gas circuit pressure sensor pressure measurement comparison value T7 into the computer monitoring host;
inputting a pressure sensor pressure measurement comparison value T′ into the computer monitoring host;
manually opening a manual carrier gas pressure reducing valve;
manually opening a standard gas pressure reducing valve;
calculating, via the carrier gas output pressure sensor, a carrier gas output pressure T0;
comparing a carrier output pressure T0 to the carrier gas output pressure sensor pressure comparison value T2; and
in response to the carrier output pressure T0 being less than or equal to the carrier gas output pressure sensor pressure comparison value T2, determining that the pressure in a carrier gas storage is insufficient, alarming the carrier gas storage, and replacing the carrier gas storage.

8. The method for using a gas analysis system of claim 7, further comprising:
calculating, via the carrier gas pressure sensor, a carrier gas pressure T4;
comparing the carrier gas pressure T4 to the carrier gas path pressure sensor pressure comparison value T6; and
in response to the carrier gas pressure T4 being not equal to the carrier gas path pressure sensor pressure comparison value T6, automatically adjusting, via an intrinsically safe PLC, a carrier gas ratio such that the carrier gas pressure T4 is equal to the carrier gas path pressure sensor pressure comparison value T6.

9. The method for using a gas analysis system of claim 8, further comprising:
calculating, via the standard gas output pressure sensor, a standard gas output pressure T1;
comparing the standard gas output pressure T1 to the standard gas output pressure sensor pressure measurement comparison value T3; and
in response to the standard gas output pressure T1 being less than or equal to the standard gas output pressure sensor pressure measurement comparison value T3, determining that the pressure in the standard gas storage is insufficient, alarming the standard gas storage, and replacing the standard gas storage.

10. The method for using a gas analysis system of claim 9, further comprising:
calculating, via the standard gas circuit pressure sensor, a standard gas circuit pressure T5;
comparing the standard gas circuit pressure T5 to the standard gas path pressure sensor pressure measurement comparison value T7;
in response to the pressure measurement value of the standard gas circuit pressure T5 being not equal to the standard gas path pressure sensor pressure measurement comparison value T7, automatically adjusting, via the intrinsically safe PLC, a standard gas proportional solenoid valve opening such that the pressure measurement value of the standard gas circuit pressure T5 is equal to the standard gas path pressure sensor pressure measurement comparison value T7;
turning on a remote power control switch from the computer monitoring host;
turning on a sampling pump; and
manually adjusting a manual flow control valve to make a flow value L equal to a set point within a range of values L0.

11. The method for using a gas analysis system of claim 10, further comprising:
calculating, via the pressure sensor, a pressure measurement value T;
comparing the pressure measurement value T to the pressure sensor pressure measurement comparison value T′; and
in response to the pressure measurement value of the pressure measurement value T being less than or equal to the pressure sensor pressure measurement comparison value T′, determining that the pressure in a gas transmission pressure is insufficient, alarming the sampling pump, and replacing the sampling pump.

12. The method for using a gas analysis system of claim 11, further comprising:
selecting a calibration option on the computer monitoring host; and
setting an intrinsically safe gas chromatograph to calibration status.

13. Thee method for using a gas analysis system of claim 12, further comprising
selecting a gas sampling port corresponding to any pipeline to be analyzed from the computer monitoring host;
opening an intrinsically safe solenoid valve;
analyzing, via the intrinsically safe gas chromatograph, the sample gas;
automatically opening another intrinsically safe solenoid valve corresponding to the gas sampling port;
analyzing another sample gas until all gas in the gas sampling port is analyzed;
reading a flowmeter reading from the computer monitoring host at any time; and
tracking a process.

14. The method for using a gas analysis system of claim 13, further comprising:
selecting the carrier gas output pressure sensor pressure comparison value T2 to be 2 Mpa;
selecting the carrier gas path pressure sensor pressure comparison value T6 to be 0.4 Mpa;
selecting the standard gas output pressure sensor pressure measurement comparison value T3 to be 2 Mpa;
selecting the standard gas path pressure sensor pressure measurement comparison value T7 to be 0.1 Mpa; and
selecting the pressure sensor pressure measurement comparison value T′ to be 0.005 Mpa.

15. The method for using a gas analysis system of claim 13, further comprising:
selecting the carrier gas output pressure sensor pressure comparison value T2 from a range of 1.5 Mpa to 2.5 Mpa;
selecting the carrier gas path pressure sensor pressure comparison value T6 from a range of 0.3 Mpa to 0.5 Mpa;
selecting the standard gas output pressure sensor pressure measurement comparison value T3 from a range of 1.5 Mpa to 2.5 Mpa;
selecting the standard gas path pressure sensor pressure measurement comparison value T7 from a range of 0.02 Mpa to 0.2 Mpa;
selecting the pressure sensor pressure measurement comparison value T′ from a range of 0.002 Mpa to 0.01 Mpa; and
selecting the range of values L0 from a range of 0.5 L/min to 8 L/min.

16. The method for using a gas analysis system of claim 15, further comprising:
selecting the carrier gas output pressure sensor pressure comparison value T2 to be 2 Mpa;
selecting the carrier gas path pressure sensor pressure comparison value T6 to be 0.4 Mpa;
selecting the standard gas output pressure sensor pressure measurement comparison value T3 to be 2 Mpa;
selecting the standard gas path pressure sensor pressure measurement comparison value T7 to be 0.1 Mpa; and
selecting the pressure sensor pressure measurement comparison value T′ to be 0.005 Mpa.
